(19) United States
(12) Patent Application Publication
(10) Pub. No.: US 2013/0052647 A1
(43) Pub. Date: Feb. 28, 2013

(54) METHODS FOR FRACTIONATING AND PROCESSING MICROPARTICLES FROM BIOLOGICAL SAMPLES AND USING THEM FOR BIOMARKER DISCOVERY

(71) Applicants: Marianna Goldrick, Austin, TX (US); Lance Ford, Austin, TX (US)

(72) Inventors: Marianna Goldrick, Austin, TX (US); Lance Ford, Austin, TX (US)

(21) Appl. No.: 13/578,464

(22) PCT Filed: Feb. 10, 2011
(86) PCT No.: PCT/US11/24385

§ 371 Date: Nov. 13, 2012

Related U.S. Application Data
(60) Provisional application No. 61/303,123, filed on Feb. 10, 2010.

(51) Int. Cl. C12Q 1/68; C07K 1/14; C12S 3/18; C07H 1/08; C12Q 1/02; C12N 5/00

(52) U.S. Cl. 435/6.12; 435/29; 435/317.1; 435/271; 536/23.1; 530/412; 422/261

(57) ABSTRACT

Described herein are methods, devices, and compositions for fractionation and processing of microparticles from biological samples, and to methods for obtaining and using the microparticles for biomarker discovery. Biological samples include cell-free fluids, for example blood plasma, blood serum, cerebrospinal fluid, urine, and saliva, as well as conditioned media. Conditioned media is the liquid growth media used to propagate cells in vitro. Purification of microparticles from cell-free fluids is challenging, typically accomplished by prolonged ultracentrifugation. Described herein is an alternative method for efficiently harvesting and processing microparticles from cell-free fluids and from conditioned media. Embodiments described herein relate to use of the microparticles and their contents recovered from conditioned media derived from propagation of human and animal cells, as a source of biomarkers for diagnosis and prognosis of diseases and pathological conditions.

### BACKGROUND OF THE INVENTION

1. Field of the Invention

The present invention relates to molecular biology and clinical diagnostics, specifically to methods, devices, and compositions for fractionation and processing of microparticles from biological samples, and to methods for obtaining and using the microparticles for biomarker discovery. Biological samples include cell-free fluids, for example blood plasma, blood serum, cerebrospinal fluid, urine, and saliva, as well as conditioned media. Conditioned media is the liquid growth media used to propagate cells in vitro. Purification of microparticles from cell-free fluids is challenging, typically accomplished by prolonged ultracentrifugation. We have developed an alternative method for efficiently harvesting and processing microparticles from cell-free fluids and from conditioned media. The invention also generally relates to use of the microparticles and their contents recovered from conditioned media derived from propagation of human and animal cells, as a source of biomarkers for diagnosis and prognosis of diseases and pathological conditions.

2. Description of the Relevant Art

There is a growing appreciation of the biological role of different types of membrane-bound microparticles (MPs) shed by cells and tissues. As used in this application, the term “microparticles” refers to all types of membrane-bound particles released by cells and platelets, including microvesicles, microparticles, tumor microvesicles, exosomes, ectosomes, platelet dust, apoptotic bodies, etc. These particles, which may be roughly spherical or irregular in shape, differ according to size and origin, for example apoptotic bodies range in size from one to several micrometers and are shed from cells undergoing apoptosis (the process of “programmed cell death”), while microvesicles, which are smaller than 1 μm, are thought to bud off directly from the plasma membrane of healthy cells. Exosomes are even smaller, on the order of 30-100 nanometers (nm), and are released from multivesicular bodies by the process of exocytosis. The distinction between different types of MPs is not always clear. For example the term apoptotic bodies has been used synonymously with microparticles.

MPs contain integral membrane proteins reflecting their cells of origin, and bioactive molecules including mRNA, microRNA and proteins. Many recent publications describe between-cell delivery of biological signals mediated by proteins and RNA contained in MPs (see above references). MPs are thought to be produced by all or most types of cells and tissues. MPs shed from cells and solid tissues can enter the circulation and occur in cell-free fluids, for example in the non-cellular fraction of blood known as plasma or serum. MPs are also shed in abundance into conditioned media, which is the media used to propagate cells in vitro, including eukaryotic and prokaryotic cells, mammalian cells, and especially including human cells. Large quantities of MPs are shed into conditioned media from malignant cells, from which the shedding of MPs may be increased compared to non-malignant cells. MPs shed from tumors may enter the circulation and mediate growth of tumors, for example by inducing growth of blood vessels that deliver nutrients to support tumor growth. MPs detected in the circulating blood of cancer patients are potential sources of diagnostic and prognostic biomarkers. Current research is focused on gaining a better understanding of the role of MPs in delivery of biological signals between cells and tissues, in both disease and health, and in identifying MP-based biomarkers. Improved methods for concentrating MPs from conditioned media and from cell-free body fluids will allow samples to be processed more rapidly and at a lower cost. This will permit detection of low-abundance MP-derived analytes, including RNAs and proteins, and increase the sensitivity of assays aimed at quantitative detection of MPs for clinical purposes, including biomarker discovery. The term “biomarkers” refers to molecules of biological origin (for example RNA, DNA, and proteins) derived from patient samples, whose levels have diagnostic and/or prognostic value.

A limitation in MP-based research is that current methods for recovering these particles from the relatively large volumes of fluids in which they occur, are laborious, time-consuming, and require expensive specialized equipment. The currently used methods for concentrating MPs from blood or culture media involve sequential centrifugation of liquid samples at increasing relative centrifugal force (rcf) to first remove intact cells and larger cell fragments and debris by low-speed centrifugation, followed by centrifugation at higher rcf to pellet the particles of interest. The rcf used to recover particles varies according to their size. For example, in one study, conditioned media was centrifuged for 10 min at 800×g to remove dead cells and cell debris, then further centrifuged for 20 min at 16,000×g to pellet the relatively large apoptotic bodies. In another study, microvesicles shed by cultured tumor cells were isolated by centrifuging the culture media for 30 min at 2,000 rpm to remove cell debris, followed by centrifugation at 20,000 rpm for 2 hours to pellet the MPs. To recover smaller particles, even higher ref is required, for example one study showed that for isolation of microvesicles it is widely accepted that ultracentrifugation should be performed at 100,000×g from 20-60 minutes and that in order to recover MPs smaller than 100 nm (the so-called exosomes), sucrose gradient ultracentrifugation is required.

Concentrating MPs by centrifugation requires expensive equipment. For example Beckman ultracentrifuges retail for around $60,000-$70,000 and the rotors required for their use list for $20,000-$23,000. Such equipment is not routinely available, especially in clinical labs and small research labs. These considerations argue for the need for better methods to facilitate processing of liquid samples to purify MPs. Once this hurdle has been passed, rapid gains can be expected in basic and applied research needed to exploit the use of MPs for therapeutic and diagnostic applications. The invention described herein overcomes the requirement for sequential centrifugation and for ultracentrifugation to purify MPs. The invention further describes the use of MPs from conditioned media for discovery of biomarkers. The biomarkers will enable use of MPs produced by patient cells propagated in vitro for improved diagnosis and treatment of disease.

Several research groups have reported identification of candidate biomarkers in RNA, microRNA, or proteins extracted from MPs present in human cell-free bodily fluids, especially blood plasma and serum. However, size-fractionated MPs recovered from conditioned media from patients' cultured cells have not been described as source of biomarkers. A major technical advantage of identifying RNA biomarkers in MPs compared to using solid tissues or circulating blood cells or cells cultured in vitro as the source tissue, is that RNA in MPs is expected to be much more stable in the transcriptionally inactive particles shed from cells (MPs), compared to their status in metabolically active tissues and cells, where rapid up-and down-regulation of specific RNA levels has been well-documented. Also, RNA in MPs is protected from the nucleases present in high concentrations in conditioned media and in cell-free body fluids such as blood plasma and urine. The potential of MPs to serve as biomarkers and potentially also as therapeutic agents will require considerable basic research followed by major development efforts to translate new findings into clinical assays and products. An important first step toward this goal is to create methods that allow rapid, economical, high-yield purification of MPs and their contents, especially RNA and proteins.

In light of the above considerations and notwithstanding the acknowledged ambiguity in the nomenclature used to describe the various types and origins of cell-derived microparticles, it can be reasonably concluded that MPs can be classified according to size into at least 3 categories: a. MPs greater than approximately 1 micron in diameter (for example apoptotic bodies); b. MPs smaller than 1 micron but larger than approximately 0.1 micron in diameter (for example microvesicles); c. MPs smaller than 0.1 micron in diameter (for example exosomes); and further, that MPs can be fractionated from fluids containing mixtures of different types of MPs by sequential centrifugation of the fluids at increasing centrifugal forces of approximately 800-2,000×g, 16,000-20,000×g, and 80,000-100,000×g. Further, MPs can be recovered from primary and long-term in vitro cultures of mammalian cells, including human cells. “Primary” cell cultures are those in which the cells originated from living tissues removed from an animal, whereas long-term cultures relate to populations of cells surviving after the primary culture has been “passaged” many times. “Passaging” cultured cells means removing a subset of growing cells, typically along with some of the conditioned media in which they have been growing, and transferring them to a new vessel, along with fresh culture media; after transfer, the cells continue to grow and divide (a process known as “expansion”). Typically, after a set number of passages, many of the primary cells and their progeny undergo the process of senescence, meaning they fail to continue to divide; however, a relatively few minority of cells may survive senescence and continue to grow and divide, thus establishing a long-term immortal cell line derived from a particular primary culture. Examples of tissues used as source of primary cells for in vitro culture are cells from solid tumors or malignancies of the blood, as well as healthy tissues including blood, endothelial cells, skin fibroblasts, epithelial tissue, etc.

MPs isolated from conditioned media from primary and/or long-term culture of a patient's cells have potential to serve as source of biomarkers. When the primary cells are pathological in origin, the MPs may serve as a source for clinically useful biomarkers for diagnosis and/or treatment of the pathological condition, for example, malignancy. It is contemplated that MPs can be harvested from conditioned media obtained from primary and/or long-term culture of an individual's cells, and that contents of the MPs, including RNA, microRNA, and proteins, can be extracted from the MPs to provide clinically useful information. It is further contemplated that the information content of the MPs will be more useful when the naturally occurring heterogeneous mixtures of MPs are first fractionated according to size, prior to extracting their contents for analysis. It is further contemplated that analysis of contents of MPs fractionated according to size will improve the ability to discover MP-derived biomarkers and apply them for clinical use.

Many experimental approaches are contemplated for biomarker discovery and use in MPs obtained from conditioned media. One approach is comparison of the levels of biomolecules in MPs derived from patient samples, with the levels of the same biomolecules derived from healthy individuals. Another approach is investigation of MP content in temporal space, which may itself consist of at least 2 types. One type of temporal analysis is analysis of MPs from conditioned media collected over a time-course, for example collected after shorter and longer periods of culture of primary cells derived from a patient's tumor. Another type of temporal analysis is analysis of MPs from primary cells obtained from tissues harvested sequentially at different times during the course of disease, for example from a needle biopsy of a newly-diagnosed malignancy and from a subsequent biopsy of a tumor from the same patient after treatment. Analysis of sequential MP samples from conditioned media samples obtained over temporal space is expected to lead to identification of associative patterns that can be developed into clinically useful biomarkers. Another approach for biomarker discovery in MPs harvested from conditioned media is to compare contents of MPs recovered from cells of different lineages, for example malignant tumor cells, endothelial cells, or blood leukocytes, that may grow out of a single tissue isolate. Yet another approach for MP-based biomarker discovery and use is to analyze MPs recovered from experimentally treated cells, for example cells treated to induce apoptosis. One of the most straightforward treatments of cultured cells to induce apoptosis is to grow them under “serum starvation” conditions, that is, in media from which the usual component of fetal bovine serum (typically added to basic liquid growth media to a level of ˜10%-20%) is withheld. These approaches are not mutually exclusive and can be combined. For example, one could carry out temporal analysis of MPs recovered from conditioned media from treated cells of several lineages derived from a single tumor. For all of these approaches, the promise of identifying new biomarkers for clinical use will be more easily realized by using size-fractionated MPs, rather than using complex mixtures of MPs of divergent sizes (which reflect their divergent biological origins). Better methods to fractionate and process conditioned media-derived MPs will facilitate MP-related basic research, leading to significant clinical benefits. The present disclosure overcomes current limitations in obtaining and processing size-fractionated MPs from heterogeneous samples.

### SUMMARY OF THE INVENTION

In one embodiment, a method for concentrating and fractionating microparticles according to their size from a liquid sample, comprises passing the sample sequentially through at least two filters having different pore sizes, wherein said filters are effective to trap membrane-bound particles of different sizes from the liquid sample. The filters may be contained in devices having inlet and outlet ports such that the devices can be attached to each other and to standard syringe(s). One or more of the filters may include a pre-filter effective to remove debris from the liquid sample which could otherwise interfere with entrapment of the membrane-bound particles.

In one embodiment, a sample is passed through a first filter having a pore size effective to trap larger particles from the liquid sample, and subsequently passed through one or more additional filters having pore size(s) effective to trap particles smaller than the larger particles from the liquid sample. The first filter may have has a pore size of about 200 nanometers (nm) and one or more of the additional filter has a pore size of about 20 nm. In another embodiment, the first filter has a pore size of about 700 nm-1,000 nm, a second filter has a pore size of about 200 nanometers (nm), and a third filter has a pore size of about 20 nm.

In an embodiment, the filters are attached to each other prior to passing the liquid sample through them, such that the sample passes through a first filter and then through one of more additional filters. The filters may be separated after passing the liquid sample through them. The separated filters with trapped particles may be processed to extract the contents of the particles. In one embodiment, the process of extracting the contents of the particles includes: passing a reagent through the separated filters, said reagent having a composition effective to disrupt the membranes of the trapped particles, thereby forming a particle lysate; collecting the particle lysate; and treating the particle lysate in a manner to purify and concentrate one or more biological components present in said lysate. Biological components that may be extracted include RNA, DNS, proteins, or combinations thereof.

In one embodiment, a method for identifying biomarkers having clinical utility for diagnosis and/or treatment of disease includes: obtaining two or more samples of tissue or cells; treating and maintaining the samples under conditions effective to allow in vitro propagation of cells in said samples in a liquid medium; recovering the liquid medium used to propagate the cells from the samples; processing the liquid medium from the samples using filters to recover membrane-bound particles present in the samples; purifying one or more biological components from particles retained on the filters from the samples; determining the levels of one or more purified biological components from the samples; comparing the levels of one or more of the purified biological components recovered from one or more samples between said samples; and determining associations between compared levels of one or more of the purified biological components recovered from one or more samples, where said associations have correlations with different physiological conditions in the one or more samples. The biological component being compared may be RNA (e.g., microRNA), DNA, proteins, or combinations thereof.

In one embodiment, a method for obtaining diagnostic or prognostic information for clinical use includes: obtaining a sample of tissue or cells from an individual; treating and maintaining the sample under conditions effective to allow in vitro propagation of cells in said samples in a liquid medium; recovering the liquid medium used to propagate the cells from the sample; processing the liquid medium from the sample using filters to recover membrane-bound particles present in the samples; purifying one or more biological components from particles retained on the filter from the sample; determining the levels of one or more purified biological components from the sample; and analyzing the levels of one or more of the purified biological components recovered from the sample to determine associations between said levels and known expected values of said components, said analysis effective to provide prognostic or diagnostic information relevant to the individual.

In one embodiment, a method for obtaining diagnostic or prognostic information for clinical use includes: obtaining a sample of cell-free bodily fluid from an individual; processing the fluid using filters to recover membrane-bound particles present in the fluid; purifying one or more biological components from particles retained on the filter from the sample; determining the levels of one or more purified biological components from the sample; and analyzing the levels of one or more of the purified biological components recovered from the sample to determine associations between said levels and known expected values of said components, said analysis effective to provide prognostic or diagnostic information relevant to the individual.

In one embodiment, a kit for fractionating MPs from biological samples, includes one or more filters and devices effective to capture said MPs from the samples, and optionally also comprising reagents for extracting, concentrating, and purifying the contents of said MPs.

In one embodiment, a method for preparing lipids and/or lipid-containing material from MPs includes: concentrating and fractionating microparticles according to their size from a liquid sample; treating the fractionated microparticles with reagent(s) effective to disrupt and solubilize their membranes; and recovering the disrupted and solubilized membrane components. The disrupted and solubilized membrane components may be further purified by treatment with nucleases and/or proteases and/or treated to remove solvents or detergents.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts an analysis of RNA extracted from MPs trapped on filters.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise.

Described herein are methods, devices and reagents for fractionating and purifying microparticles (MPs) and their contents from biological samples. A primary feature of the embodiments described herein are the use of a series of filters having suitable properties for trapping MPs of different sizes from biological samples, and devices to allow use of such filters for recovering MPs from biological samples. Examples of biological samples that may be used with said filters and devices are blood serum and blood plasma from humans and other mammalian and non-mammalian animals, as well as conditioned media used to propagate cells in culture. The MPs captured on the filters may subsequently be recovered from the filters as intact particles, said particles having potential for use as delivery agents for transferring their contents to recipient cells. Contents of potential interest that may be transferred by intact MPs are proteins, chemicals, DNA, mRNAs, microRNAs, siRNAs, and other non-coding RNAs.

As an alternative to recovering intact MPs from the filters for use as delivery agents, the MPs captured on the filters may be processed by disrupting the MPs and collecting their contents. Disruption of captured MPs may be accomplished by removing the filters along with the trapped MPs to a second vessel, for example a microfuge tube, and adding reagent(s) capable of disrupting the MP membrane and releasing its contents. Alternatively, recovery of the MP contents may be accomplished by in situ disruption of the MP's membranes without removing the filters from the device in which they are placed, for example by flushing the filters with reagent(s) effective to disrupt the captured MPs and release their contents into a separate vessel. Disruption of MPs can be accomplished by use of various reagents. For disruption of MPs and subsequent purification of their RNA and/or protein contents, single-phase reagents containing chaotropic agent(s) (such as guanidinium thiocyanate or guanidinium hydrochloride) and organic solvent (such as phenol) are especially useful. Alternative reagents such as those based on other denaturing chemicals such as urea, or on other nuclease-inactivating reagents such as proteases, may be used instead of single-phase reagents containing guanidinium and phenol. Other types of membrane-disrupting reagents familiar to those skilled in the art of molecular biology may also be used to disrupt the MPs. Further purification and concentration of the contents of disrupted MPs can be accomplished using a variety of different methods known to those skilled in the art, for example alcohol precipitation or solid-phase extraction onto silica matrices. An aspect of the process includes the use of reagents and protocols for further purifying and concentrating the contents of MPs trapped on filters. A further aspect of the process relates to use of biomolecules recovered from MPs released by cells, especially those grown in vitro, as a source for discovery of biomarkers and as source sample for use of said biomarkers for diagnosis and prognosis of pathological conditions.

In addition to recovering and using the contents of disrupted MPs, it is contemplated that recovery of the lipid monomers and other lipid-containing materials originating from the disrupted membranes of MPs, may also be useful. Such lipids or lipid-containing materials could be recovered by solubilizing the MPs in solvents such as chloroform or by disrupting the MPs in reagents containing detergents. The solvents or detergents can then be removed, for example by evaporation or by chromatography, leaving the lipids and lipid-containing materials in a more concentrated foam. It is contemplated that such lipids and lipid-containing materials will be useful for preparing liposomes for delivery of natural or synthetic molecules, especially for clinical purposes. Examples of synthetic molecules that may be delivered by MP-derived liposomes and that have utility for clinical purposes are small interfering RNA molecules (siRNAs) and synthetic DNA molecules that may encode siRNAs, and recombinant viral vectors. Examples of natural molecules that may be delivered by MP-derived liposomes and that have utility for clinical purposes are plasmids, viruses, and antibodies. Natural and synthetic molecules may be incorporated into the liposome membranes and/or into the interior space of the liposomes. In cases where it is desirable to recover lipids or lipid-containing materials derived from membranes of MPs in a substantially pure form wherein the lipids or lipid-containing materials are not mixed with the contents of the MPs, the contents of the MPs can be removed or rendered inactive by disrupting the MPs, or by treating the material comprising disrupted MPs, with reagents containing nucleases, including DNases and RNases, and/or by treating or disrupting the MPs with reagents containing proteases.

In one embodiment, a process uses filtration, instead of the currently used method of centrifugation, to directly capture MPs from liquid samples. In an embodiment, a series of two or more filters having different properties are used to allow entrapment of MPs of different sizes. Liquid samples containing mixtures of MPs that differ in their origin, sizes, and contents, may be recovered as separate populations for further analysis. In one embodiment, the filters are contained in plastic devices that provide support for the filters and that allow attachment of the filters to a reservoir that is used to contain the liquid sample prior to processing. An especially useful design for such devices is as so-called “syringe filters”, in which filters are placed over a perforated support to allow liquid to flow through the filter, and with the device having inlet and outlet ports designed such that the devices can be readily connected to each other and to standard syringes. The syringes can be of different sizes, ranging from less than 1 ml to 60 ml, to allow processing of samples of a wide range of volumes. For cases in which two or more syringe filter devices are connected to each other, they are connected such that the top filter, that is the filter in which the liquid sample is first contacted, has the largest effective pore size, enabling entrapment of the largest particles, and subsequent filters are in order of decreasing pore size, to enable successive entrapment of smaller and smaller particles. The top filter in a series of connected filters may have a pore size of >1 μM, effective to trap intact cells with minimal entrapment of smaller microparticles. To maximize recovery and size homogeneity of MPs, a liquid sample may be passed over a series of 2 or more filters, or over a single filter, and the “flow-through” liquid passing through the filter(s) may be recovered and passed once again over the same filter(s). After the liquid sample has been passed over a series of one or more filters, the filters are then disconnected and processed separately, to allow recovery of the size-fractionated MPs or their contents as separate preparations.

In one process, filters are used to harvest MPs from cells grown in tissue culture. Different types of mammalian cells are known to naturally shed MPs into the culture media. As discussed above, these MPs can serve as a source of biomarkers. Those skilled in the art of molecular biology will also appreciate that primary cells or immortalized cell lines can be engineered, using methods known to those with skill in the art, to make MPs with useful RNA and/or protein content.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Fractionation of Populations of Smaller and Larger MPs from Tissue Culture Media

Conditioned media was obtained from several human cancer cell lines (A549 lung cancer, CLL lymphoma cells, and HL60 liver cancer cells) growing as adherent cells, that is, the cells were attached to the bottom of the culture vessels. Typical volumes of conditioned media from adherent cells range from about 5 ml-15 ml per 100 mm tissue culture dish. In general, the conditioned media may be loaded into a syringe of appropriate capacity for the volume of sample, by aspirating the media into the syringe. Syringe filters having pore sizes effective to trap MPs from the conditioned media are then attached to the outlet port of the syringe. Alternatively, the plunger of the syringe may be removed, the syringe filter(s) then attached, and the conditioned media then loaded by pouring it into the barrel of the syringe and then reinserting the plunger. In either case, the end result is a syringe containing the conditioned media, with the filter(s) attached. The latter option for loading the sample into a 12 ml syringe was used in this Example.

The filters used in this Example consist of two syringe filters, each ˜25 mm in diameter, with the top filter having a pore size of ˜200 nanometers (0.2 microns), and the bottom filter having a pore size of ˜20 nanometers (0.02 microns). The syringe filters were obtained from Tisch Scientific (Village of Cleves, Ohio 45002, USA). The filter having a pore size of ˜200 nanometers was catalog #SF14499, and the filter having a pore size of ˜20 nanometers was catalog #SF15016. The syringe filter-syringe assembly was then positioned over a vessel to catch the flow-through. The plunger of the syringe was then gently depressed to apply the force needed to drive the media sample through the connected filters. After the sample had passed through the filters, they were removed from the syringe and then disconnected from each other. Residual media in the filter devices was removed by tapping the outlet ports of the devices on a paper towel. Each of the filter devices were separately processed by attaching each to a 6 ml syringe which had been preloaded with 1 ml of a single-phase reagent (BiooPure RNA Extraction Reagent, Bioo Scientific, Austin, Tex.) comprising phenol, guanidinium thiocyanate, and other components effective to disrupt cell membranes, including the membranes that delineate cellular MPs. The assemblies were positioned over 1.5 ml microcentrifuge tubes, and the plungers of the syringes slowly depressed to force the BiooPure reagent into the filter device. The assembly was tilted during delivery of the BiooPure reagent into the filter device, in order to maximize contact of the reagent with the surface of the filter. Contact of the reagent with the entire surface of the filter is confirmed visually, by presence of green color on the entire surface of the filter. Contact of the reagent with the trapped MPs causes their membranes to be disrupted, resulting in release of their contents. The plungers were then fully depressed to flush the reagent, along with the contents of the disrupted MPs, into 1.5 ml receiving tubes. The samples (lysates from size-fractionated MPs) were then mixed thoroughly by vortexing. Samples may be processed immediately to purify and concentrate the released contents of the MPs, or the samples may be stored for subsequent processing. In this Example, the samples were stored at −20 C. for several days prior to further processing.

### Example 2

### Extraction of RNA from MPs Recovered from Conditioned Media

Extraction of RNA from MPs was carried out using a proprietary reagent (BiooPure RNA Extraction Reagent) developed at Bioo Scientific, which is similar to Trizol (sold by Sigma and other vendors). Trizol has also been used for extraction of MPs captured from conditioned media on filters. Trizol and BiooPure are both single-phase reagents containing phenol and guanidinium, and the extraction protocols are similar. RNA was extracted by thawing the preparations (described in Example 1) and adding 0.1 ml of 1-bromo-3-chloropropane (purchased from Sigma Life Science Research products, cat #B9673), vortexing the prep for ˜20 sec, then centrifuging the prep in a microcentrifuge for ˜15 min at 4 C. The resulting separated aqueous phase (top phase) was transferred to a new 1.5 ml tube and mixed with 50 μg of linear polyacrylamide (Bioo Scientific), followed by mixing with 0.75 ml of isopropyl alcohol. The prep was stored at room temp for ˜15 min and then centrifuged for 15 min at 12,000 rpm at 4 C. The supernatant fluid was carefully removed and the pelleted material was washed by adding 0.6 ml of 75% ethanol (diluted in nuclease-free water), vortexing to dislodge the pellet, then re-centrifuging the prep for 10 min at 10,000 rpm at 4 C. The supernatant fluid was thoroughly removed and the pellet of RNA dissolved in 50 μL of 0.1 mM EDTA made in nuclease-free water. To aid solubilization, the prep was vortexed, then incubated for 5 min at 65 C. in a heat block, then re-vortexed and centrifuged briefly to collect all liquid at the bottom of the tube. The preparation was then stored at −20 C. until use.

### Example 3

### Quantitative Detection of microRNAs in RNA Extracted from Size-Fractionated MPs Recovered from Conditioned Media

Recovery of RNA from the MPs captured on filters from conditioned media was verified by using a commercially available microRNA-detection assay from Life Technologies Inc. This assay is based on reverse transcription followed by quantitative PCR(RT-qPCR). We have also used this assay to detect microRNA in preps from MPs recovered from human serum.

The reverse transcription step was carried out in a 7.5 μL volume containing 2.5 μL of RNA prepared as described in Example 2 along with approximately 50 units of MMLV Reverse Transcriptase (Bioo Scientific), standard buffer components, and reverse transcription primers for several microRNAs (miR-150, miR-191, and miR-337), provided in the Life Technologies assays. Reactions were incubated according to the Life Technolgies protocol. For the qPCR step, 1.5 μL of each reverse transcription reaction was used as template for duplicate amplification reactions (“technical duplicates”) of 20 μL, using the microRNA target-specific amplification primers from the Life Technologies assay according to manufacturer's instructions. Reactions were carried out using a BioRad iQ real-time instrument and Ct values recorded. Ct stands for cycle threshold, the amplification cycle number at which a detectable fluorescent signal is generated over a preset background level; in this study the instrument default value was used for the background and detection settings. The lower the Ct value, the higher the abundance of target molecule in the sample (since the more abundant the target, the fewer PCR cycles are needed to amplify it to a detectable level). Due to the exponential nature of PCR, a difference of 3.32 Ct's corresponds to a ˜10-fold difference in target abundance (since 2̂3.32˜10). We observed the following data in this experiment:

| Sample RNA | miR-150 Ct value | miR-191 Ct value | miR-337 Ct value |
|---|---|---|---|
| RNA recovered from | Technical duplicates: | Technical duplicates: | Technical duplicates: |
| MPs trapped on top | 29.76/29.69, | 24.62/24.63 | 32.07/32.39 |
| filter having larger | avg = 29.72 | Avg = 24.62 | Avg = 32.23 |
| pore size | | | |
| RNA recovered from | Technical duplicates: | Technical duplicates: | Technical duplicates: |
| MPs trapped on | 28.22/27.91 | 27.69/26.63 | 31.81/31.95 |
| bottom filter having | Avg = 28.07 | Avg = 27.16 | Avg = 31.88 |
| smaller pore size | | | |
| Negative control (no | Not detected (Ct > 45) | Not detected (Ct > 45) | Not detected (Ct > 45) |
| input cDNA in PCR | | | |
| step) | | | |

This experiment demonstrates the effectiveness of the filters for concentrating microRNA from conditioned media. Since the relative levels of specific microRNAs in fractionated MPs obtained from conditioned media have not been reported previously, there is no basis for comparison of our results to those of others. However this experiment indicates that the filters trapped different populations of MPs, because the relative levels of the 3 microRNAs differed in the MPs trapped on the filter with larger pore size compared to the filter having smaller pore size. The level of miR-191 is ˜34-fold higher than miR-150 in MPs captured on the filter with larger pore size (2̂5.1), while the level of miR-191 is only ˜1.9 fold higher than miR-150 in MPs captured on the filter with smaller pore size (2̂0.91).

### Example 4

### Fractionation and Processing of MPs from Human Serum

Human blood serum was purchased from a commercial source (Innovative Research) and approximately 5 ml of serum from a single donor was fractionated over a filter having a 20 nm pore size as described in Example 1. The filter was then flushed with RNA extraction reagent to lyse the trapped particles as described in Example 2; the lysate was recovered and saved for RNA extraction. This sample is referred to as “Serum filter”. Prior to filtration, 0.25 ml of serum was removed and mixed with RNA extraction reagent as described in Example 2 (this sample is referred to as “pre-filtration serum”). After filtration, 0.25 ml of serum that had passed through the filter was removed and mixed with RNA extraction reagent (this sample is referred to as “flow-through serum”). RNA was then extracted from the 3 samples using the method described in Example 2, and the RNA from each sample was resuspended in an equal volume (30 μL) of 0.1 mM EDTA. Equal volumes of RNA recovered from each sample was used for detection of a microRNA, miR-191, as described in Example 3. The Ct values are shown below.

| Prefiltration serum | Serum filter | Flow-through serum |
|---|---|---|
| Avg Ct miR-191: | Avg Ct miR-191: | Avg Ct miR-191: |
| 32.31 | 27.83 | 34.01 |

The ability of the filter to concentrate RNA signal from human serum is verified by comparing the mir-191 signal in RNA extracted from unfractionated serum and in the flow-through sample, with the level in RNA extracted from purified MPs. The signal in the filter sample is approximately 22-fold greater than in the prefiltered sample (2̂4.48) and 73-fold greater than in the flow-through sample (2̂6.18). The flow-through sample is depleted from miR-191 signal by ˜3.2 fold compared to the prefiltration sample (2̂1.7).

In the experiments described above, filters with captured MPs were processed by flushing them with RNA extraction reagent, which immediately disrupts the MN and stabilizes their RNA. While this procedure will be useful for serum biomarker discovery, for use as delivery vehicles, the MPs would need to be recovered as intact particles. A further embodiment is to recover intact MPs after their entrapment on filters. To recover intact MPs, the filters with MPs could be removed to vessels containing a physiological buffer such as PBS and vortexed to release the MPs, or the filters with trapped MPs could be back-flushed with a physiological buffer such as PBS to release the intact trapped particles.

### Example 5

### Analysis of RNA from Conditioned Media on Agilent Bioanalyzer

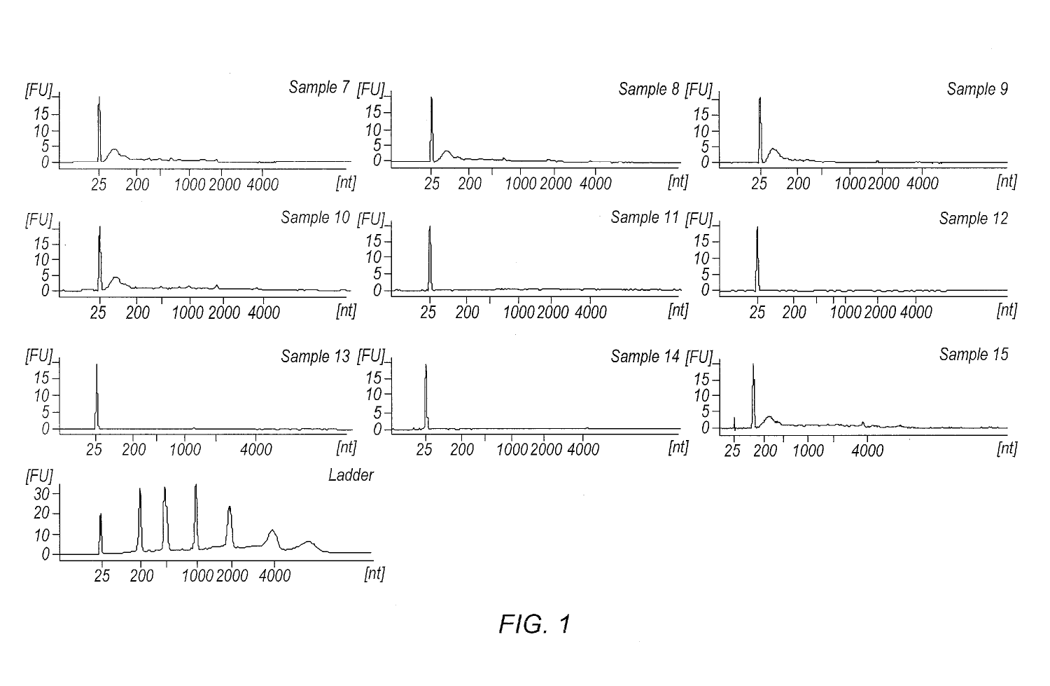

RNA was extracted as described in Example 2, from MPs trapped on filters from conditioned media and from the corresponding flow-through, and analyzed on an Agilent Bioanalyzer as shown in FIG. 1. This instrument uses capillary electrophoresis to separate RNA samples according to size. In the electropherograms shown below, the concentration of RNA in the samples is reflected by the height of the peaks (the higher concentration, the higher the peak height) and the size of the RNA is reflected in the position of the peak along the X-axis (the larger the RNA, the further it migrates in the right-hand direction). The small “hump” seen in the lower left-hand corner of the electropherogram traces in Samples 7, 8, 9, and 10 in the FIG. 2 is the RNA recovered from MPs trapped on 20 nanometer filters from 4 different conditioned media samples. Samples 11, 12, 13, and 14 show material extracted from the corresponding flow-through conditioned media. The lack of the peak in Samples 11-14 indicate that the filters were highly effective for recovering RNA from the conditioned media samples. The position of the peak near the left-hand side of the X-axis, and the lack of peaks further to the right, shows that all of the RNA detectable by this instrument in RNA retained on the filters was small RNA, of a size range centered around ˜100 bases (as calibrated by comparison of the peak positions to the molecular size marker “ladder” shown in the last panel).

### Example 6

### Use of Biomolecules Recovered from MPs for Biomarker Discovery

It is contemplated that RNA recovered from fractionated MPs obtained from different samples of cell-free bodily fluids will be used as input for determination of the relative levels of multiple different microRNAs in the samples (a process known as “microRNA profiling”). Comparison of microRNA profiles between different types of samples will allow associations to be made between microRNA profiles and phenotypic differences between the samples. For example, microRNA profiles in RNA extracted from size-fractionated MPs obtained from cell-free bodily fluids from healthy individuals, can be compared with microRNA profiles in RNA extracted from size-fractionated MPs obtained from cell-free bodily fluids from individuals known or suspected of having pathological condition(s), and appropriate analyses carried out to identify differences in microRNA profiles that can be correlated with specific pathological condition(s). In a similar manner, it is contemplated that RNA recovered from fractionated MPs obtained from different samples of conditioned media derived from primary or long-term cultures of cells grown in vitro will be used as input for determination of the relative levels of multiple different microRNAs in the samples. Comparison of microRNA profiles between different types of conditioned media samples will allow associations to be made between the microRNA profiles and phenotypic differences of the individuals from whom the cells that were used to generate the conditioned media were obtained. For example, microRNA profiles in RNA extracted from size-fractionated MPs obtained from conditioned media recovered from cells cultured from healthy individuals, can be compared with microRNA profiles in RNA extracted from size-fractionated MPs obtained from conditioned media recovered from cells cultured from individuals known or suspected of having pathological conditions, and appropriate analyses carried out to identify differences in microRNA profiles that can be correlated with specific pathological conditions.

In a similar manner, it is further contemplated that proteins and other types of nucleic acid, including DNA and messenger RNA (mRNA) recovered from fractionated MPs obtained from different samples of cell-free bodily fluids and conditioned media will be used as input for determination of the relative levels of multiple different proteins and other types of nucleic acids. Comparison of levels of proteins and other types of nucleic acids between different types of samples will allow associations to be made between their levels and phenotypic differences between the samples. For example, levels of proteins and/or mRNA extracted from size-fractionated MPs obtained from cell-free bodily fluids from healthy individuals or from conditioned media derived from the cultured cells of healthy individuals, can be compared with levels of proteins and/or mRNA extracted from size-fractionated MPs obtained from cell-free bodily fluids or conditioned media derived from the cultured cells of individuals known or suspected of having a pathological condition, and appropriate analyses carried out to identify differences in microRNA profiles that can be correlated with specific pathological condition(s). In all of the above examples, it is contemplated that observed differences in levels of analytes between samples can be validated for use as biomarkers.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may in some cases be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method for concentrating and fractionating microparticles according to their size from a liquid sample, comprising passing the sample sequentially through at least two filters having different pore sizes, wherein said filters are effective to trap membrane-bound particles of different sizes from the liquid sample.

2. The method of claim 1, wherein the filters are contained in devices having inlet and outlet ports such that the devices can be attached to each other and to standard syringe(s).

3. The method of claim 1, wherein one or more of the filters contains a pre-filter effective to remove debris from the liquid sample which could otherwise interfere with entrapment of the membrane-bound particles.

4. The method of claim 1, wherein the sample is passed through a first filter having a pore size effective to trap larger particles from the liquid sample, and subsequently passed through one or more additional filters having pore size(s) effective to trap particles smaller than the larger particles from the liquid sample.

5. The method of claim 4, wherein the first filter has a pore size of about 200 nanometers (nm) and one or more of the additional filter has a pore size of about 20 nm.

6. The method of claim 4, wherein the first filter has a pore size of about 700 nm-1,000 nm, a second filter has a pore size of about 200 nanometers (nm), and a third filter has a pore size of about 20 nm.

7. The method of claim 1, wherein the filters are attached to each other prior to passing the liquid sample through them, such that the sample passes through a first filter and then through one of more additional filters.

8. The method of claim 7, further comprising separating the filters after passing the liquid sample through them.

9. The method of claim 8, wherein the separated filters with trapped particles are processed to extract the contents of the particles.

10. The method of claim 8, wherein the separated filters are processed by the process comprising:
passing a reagent through the separated filters, said reagent having a composition effective to disrupt the membranes of the trapped particles, thereby forming a particle lysate;
collecting the particle lysate; and
treating the particle lysate in a manner to purify and concentrate one or more biological components present in said lysate.

11. The method of claim 10, wherein the biological component is RNA.

12. The method of claim 10, wherein the biological component is protein.

13. The method of claim 10, wherein the biological component is DNA.

14. A method for identifying biomarkers having clinical utility for diagnosis and/or treatment of disease, comprising:
obtaining two or more samples of tissue or cells;
treating and maintaining the samples under conditions effective to allow in vitro propagation of cells in said samples in a liquid medium;
recovering the liquid medium used to propagate the cells from the samples;
processing the liquid medium from the samples using filters to recover membrane-bound particles present in the samples;
purifying one or more biological components from particles retained on the filters from the samples;
determining the levels of one or more purified biological components from the samples;
comparing the levels of one or more of the purified biological components recovered from one or more samples between said samples; and
determining associations between compared levels of one or more of the purified biological components recovered from one or more samples, where said associations have correlations with different physiological conditions in the one or more samples.

15. The method of claim 14, wherein processing the liquid medium from the samples using filters comprises comprising passing the samples sequentially through at least two filters having different pore sizes, wherein said filters are effective to trap membrane-bound particles of different sizes from the liquid sample.

16. The method of claim 14, wherein purifying one or more biological components from particles retained on the filters comprises:
passing a reagent through the separated filters, said reagent having a composition effective to disrupt the membranes of the trapped particles, thereby forming a particle lysate;
collecting the particle lysate; and
treating the particle lysate in a manner to purify and concentrate one or more biological components present in said lysate.

17. The method of claim 14, wherein the biological component being compared is RNA.

18. The method of claim 14, wherein the RNA being compared is microRNA.

19. The method of claim 14, wherein the biological component being compared is protein.

20. The method of claim 14, wherein the biological component being compared is DNA.

21. A method for obtaining diagnostic or prognostic information for clinical use, comprising:
obtaining a sample of tissue or cells from an individual;
treating and maintaining the sample under conditions effective to allow in vitro propagation of cells in said samples in a liquid medium;
recovering the liquid medium used to propagate the cells from the sample;
processing the liquid medium from the sample using filters to recover membrane-bound particles present in the samples;
purifying one or more biological components from particles retained on the filter from the sample;
determining the levels of one or more purified biological components from the sample; and
analyzing the levels of one or more of the purified biological components recovered from the sample to determine associations between said levels and known expected values of said components, said analysis effective to provide prognostic or diagnostic information relevant to the individual.

22. A method for obtaining diagnostic or prognostic information for clinical use, comprising the sequential steps of:
obtaining a sample of cell-free bodily fluid from an individual;
processing the fluid using filters to recover membrane-bound particles present in the fluid;
purifying one or more biological components from particles retained on the filter from the sample;
determining the levels of one or more purified biological components from the sample; and
analyzing the levels of one or more of the purified biological components recovered from the sample to determine associations between said levels and known expected values of said components, said analysis effective to provide prognostic or diagnostic information relevant to the individual.

23. A kit for fractionating MPs from biological samples, comprising one or more filters and devices effective to capture said MPs from the samples, and optionally also comprising reagents for extracting, concentrating, and purifying the contents of said MPs.

24. A method for preparing lipids and/or lipid-containing material from MPs comprising:
concentrating and fractionating microparticles according to their size from a liquid sample;
treating the fractionated microparticles with reagent(s) effective to disrupt and solubilize their membranes; and
recovering the disrupted and solubilized membrane components.

25. The method of claim 24, wherein the disrupted and solubilized membrane components are further purified by treatment with nucleases and/or proteases.

26. The method of claim 24, wherein the disrupted and solubilized membrane components are further treated to remove solvents or detergents.
